(19) United States
(12) Patent Application Publication
(10) Pub. No.: US 2019/0358042 A1
(43) Pub. Date: Nov. 28, 2019

(54) Tapered Osteochondral Implant

(71) Applicant: In2Bones USA, LLC, Memphis, TN (US)

(72) Inventors: Alan G. Taylor, Memphis, TN (US); Rebecca Hawkins Wahl, Escondido, CA (US)

(73) Assignee: In2Bones USA, LLC, Memphis, TN (US)

(21) Appl. No.: 15/989,975

(22) Filed: May 25, 2018

(51) Int. Cl. A61F 2/30; A61F 2/46; A61B 17/16; A61F 2/00

(52) U.S. Cl. CPC A61F 2/30756; A61F 2/4618; A61B 17/1604; A61F 2/0095; A61F 2002/3023; A61F 2002/4659; A61F 2310/00023; A61F 2310/00029; A61F 2310/00059; A61F 2310/00329; A61F 2002/30616; A61F 2002/30327

(57) ABSTRACT

An apparatus and methods are provided for a tapered implant for treating osteochondral defects. The tapered implant comprises a top portion that includes a shape that approximates an osteochondral surface to be replaced. A bottom portion of the tapered implant is configured to be implanted into a hole drilled in bone. A cylindrical sidewall of the tapered implant has a diameter that decreases from a first diameter of the top portion to a second diameter of the bottom portion. The tapered implant comprises any monophasic synthetic material suitable for implantation into bone, including any of silicone, bioglass, peek, polyethylene, titanium, and cobalt chrome.

### FIELD

Embodiments of the present disclosure generally relate to the field of surgical implants. More specifically, embodiments of the disclosure relate to an apparatus and methods for a tapered implant for treating osteochondral defects.

### BACKGROUND

Articular cartilage is a smooth, white tissue which covers the ends of bones where they come together to form joints in humans and many animals so as to facilitate articulation of the joints and protect and cushion the bones. Cartilage may become damaged, however, due to abrupt trauma or prolonged wear. A number of surgical techniques have been developed to treat damaged cartilage. Restoring articular cartilage is known to relieve pain and facilitate better joint function, as well as potentially delaying or preventing an onset of arthritis. One surgical technique comprises transplantation of a healthy osteochondral graft so as to replace damaged cartilage and encourage new cartilage growth.

Osteochondral grafting typically involves removing cartilage and bone tissue of a defect site by routing to create a cylindrical bore. A tissue scaffold such as a cylindrical cartilage and subchondral bone plug graft is harvested and then implanted into the bore of the routed defect site. Healing of the graft bone to host bone results in fixation of the plug graft to the surrounding host region.

The plug graft may be an autograft taken from another body region of less strain, such as the hip, skull, or ribs, or the plug graft may be an allograft harvested from bone taken from other people that is frozen and stored in tissue banks. In some instances, the plug graft may be a xenograft that is harvested from animals of a different species. Moreover, many grafting procedures utilize a variety of natural and synthetic tissue scaffolds, with or instead of bone, such as collagen, silicone, acrylics, hydroxyapatite, calcium sulfate, ceramics, and the like, which may be press-fit into the osteochondral hole at a patient's defect area. As such, there is an ongoing need for the development of osteochondral grafting capabilities such as that found in, for example, treating damage to articular cartilage in joints. Provided herein are embodiments and methods for a tapered monophasic implant for treating osteochondral defects.

### SUMMARY

An apparatus and methods are provided for a tapered implant for treating osteochondral defects. The tapered implant comprises a top portion that includes a shape that approximates an osteochondral surface to be replaced. A bottom portion of the tapered implant is configured to be implanted into a hole drilled in bone. A cylindrical sidewall of the tapered implant has a diameter that decreases from a first diameter of the top portion to a second diameter of the bottom portion. The tapered implant comprises any monophasic synthetic material suitable for implantation into bone, including any of silicone, bioglass, peek, polyethylene, titanium, and cobalt chrome. In some embodiments, one or more tapered implants are included in a sterile instrument kit for repairing osteochondral defects in various bone joint locations in a patient's body. The sterile instrument kit includes instruments that are configured for implanting the one or more tapered implants into the patient's body, such that the implant is flush or slightly proud of a surrounding native cartilage surface. The instruments include any one or more of an implant inserter, a guidewire, a cannulated reamer, a punch, and a size gauge, as described herein.

In an exemplary embodiment, an implant for treating osteochondral defects comprises: a cylindrical member comprised of a monophasic synthetic material; a top portion comprising a first diameter; a bottom portion comprising a second diameter; and a tapered sidewall portion disposed between the top portion and the bottom portion.

In another exemplary embodiment, the tapered sidewall portion includes a diameter that decreases from the first diameter to the second diameter. In another exemplary embodiment, the tapered sidewall portion comprises a degree of tapering that is configured to prevent the implant from subsiding into the hole drilled in bone. In another exemplary embodiment, the cylindrical member includes a surface area ranging between substantially 6 square inches and substantially 17 square inches.

In another exemplary embodiment, the monophasic synthetic material includes any one of silicone, bioglass, peek, polyethylene, titanium, and cobalt chrome. In another exemplary embodiment, the first diameter and the second diameter are selected according to a location in a patient that is to be treated. In another exemplary embodiment, a height of the cylindrical member is substantially 10 millimeters (mm), and the first diameter ranges between substantially 5 mm and substantially 10 mm.

In another exemplary embodiment, the top portion includes a shape configured to approximate an osteochondral surface to be replaced. In another exemplary embodiment, the shape includes a curvature of the top portion that approximates the curvature of the osteochondral surface to be replaced. In another exemplary embodiment, the curvature is either convex or concave so as to match the anatomy of the osteochondral surface.

In another exemplary embodiment, the implant further comprises a rounded periphery that joins the top portion and the tapered sidewall portion, the rounded periphery providing a smooth contact surface to surrounding tissues. In another exemplary embodiment, the implant further comprises a rounded periphery that joins the tapered sidewall portion and the bottom portion, the rounded periphery providing a smooth transition surface between the tapered sidewall portion and the bottom portion.

In an exemplary embodiment, a sterile instrument kit for repairing osteochondral defects comprises: one or more tapered implants configured to treat osteochondral defects in various bone joint locations in a patient's body, the one or more tapered implants each comprising monophasic synthetic material; and a multiplicity of instruments including any one or more of an implant inserter, a guidewire, a cannulated reamer, a punch, and a size gauge, the multiplicity of instruments being configured for implanting the one or more tapered implants into the patient's body such that the implant is flush or slightly proud of a surrounding native cartilage surface.

In another exemplary embodiment, the one or more tapered implants and the multiplicity of instruments are packaged together in an exterior container suitable for delivery to a practitioner. In another exemplary embodiment, the one or more tapered implants are stored in a first sterile container. In another exemplary embodiment, any one or more of the guidewire, the cannulated reamer, the punch, and the implant inserter are stored in a second sterile container. In another exemplary embodiment, the size gauge is stored in a third sterile container.

In an exemplary embodiment, a method for a sterile instrument kit for repairing osteochondral defects comprises: configuring one or more tapered implants to treat osteochondral defects in various bone joint locations in a patient's body; and combining the one or more tapered implants with a multiplicity of instruments configured for implantation of the one or more tapered implants into the patient's body, the multiplicity of instruments including at least a guidewire, a cannulated reamer, a punch, and a size gauge.

In another exemplary embodiment, configuring comprises forming the one or more tapered implants of a monophasic synthetic material, including any or one of silicone, bioglass, peek, polyethylene, titanium, and cobalt chrome. In another exemplary embodiment, configuring comprises forming the one or more tapered implants such that the diameters of a top portion of the one or more tapered implants range from substantially 5 mm to substantially 10 mm. In another exemplary embodiment, combining further comprises: storing the one or more tapered implants in a first sterile container; storing any one or more of the guidewire, the cannulated reamer, the punch, and an implant inserter in a second sterile container; and storing the size gauge in a third sterile container.

### BRIEF DESCRIPTION OF THE DRAWINGS

The drawings refer to embodiments of the present disclosure in which:

FIG. 1 illustrates an isometric view of an exemplary embodiment of a tapered monophasic implant for treating osteochondral defects, in accordance with the present disclosure;

FIG. 2 illustrates a side view of an exemplary embodiment of a tapered monophasic implant having a relatively wide diameter;

FIG. 3 illustrates a side view of an exemplary embodiment of a tapered monophasic implant having a relatively narrow diameter;

FIG. 4 illustrates a side plan view of the tapered monophasic implant of FIG. 2;

FIG. 5 illustrates an exemplary use environment comprising an exemplary embodiment of a tapered monophasic implant that is press-fit into an osteochondral hole in a 1^{st} metatarsal bone;

FIG. 6 illustrates an exemplary embodiment of a sterile instrument kit for treating damaged cartilage joints according to the present disclosure;

FIG. 7A illustrates an exemplary embodiment of a punch that may be included in the sterile instrument kit of FIG. 6; and

FIG. 7B illustrates the punch of FIG. 7A mounted onto a guidewire for the purpose of directing a distal blade of the punch to a damaged location within a bone joint.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first implant,” may be made. However, the specific numeric reference should not be interpreted as a literal sequential order but rather interpreted that the “first implant” is different than a “second implant.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Cartilage that facilitates articulation of the joints and protects and cushions bones can become damaged due to abrupt trauma or prolonged wear. A number of surgical techniques have been developed to treat damaged cartilage, thereby relieving pain and facilitating better joint function. One surgical technique includes transplantation of a healthy osteochondral graft to replace damaged cartilage and encourage new cartilage growth. Many grafting procedures utilize a variety of natural and synthetic tissue scaffolds, with or instead of bone, such as collagen, silicone, acrylics, hydroxyapatite, calcium sulfate, ceramics, and the like, which may be implanted into an osteochondral hole bored at a patient's defect area. As such, there is an ongoing need for the development of osteochondral grafting capabilities such as that found in, for example, treating damage to articular cartilage in joints. Provided herein are embodiments and methods for a tapered monophasic implant for treating osteochondral defects.

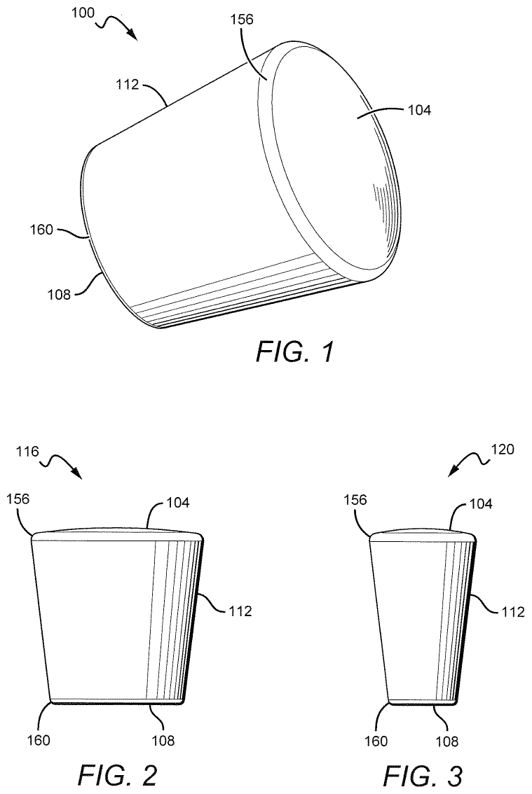

FIG. 1 illustrates an exemplary embodiment of a tapered monophasic implant 100 for treating osteochondral defects in accordance with the present disclosure. In general, the implant 100 includes a top portion 104 and a bottom portion 108 that share a cylindrical sidewall 112 extending therebetween. The implant 100 is configured to be press-fit into an osteochondral hole bored at a patient's defect area. The top portion 104 includes a shape that approximates an osteochondral surface to be replaced. The bottom portion 108 is configured to be implanted into the osteochondral hole drilled into the patient's bone. The implant 100 comprises any monophasic synthetic material suitable for implantation into bone, including any of silicone, bioglass, peek, polyethylene, titanium, and cobalt chrome.

As shown in FIGS. 2-3, the implant 100 may be implemented with a range of diameters that facilitate using the implant 100 to treat osteochondral defects in various bone joint locations in the human body, such as by way of non-limiting example, a femoral condyle, a humeral head, a talus, a capitellum of the elbow, as well as any of the metatarsal and phalangeal joints. As such, FIG. 2 illustrates a side view of an exemplary embodiment of a tapered monophasic implant 116 having a relatively wide diameter, and FIG. 3 shows a side view of an exemplary tapered monophasic implant 120 having a relatively narrow diameter. It is contemplated, however, that the overall size of the implant 100 is to be selected according to the particular bone joint to be treated.

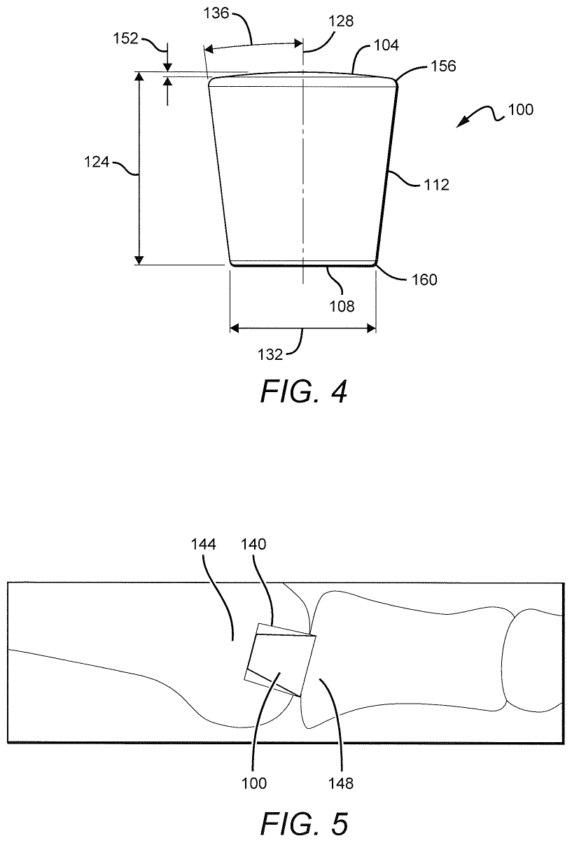

As best shown in FIG. 4, the implant 100 possesses a height 124 along a longitudinal axis 128 of the implant and a bottom diameter 132 centered on the longitudinal axis 128. The height 124 extends from the bottom portion 108 to the highest region of the top portion 104, such as the region of the top portion 104 around the longitudinal axis 128. In one embodiment, the height 124 is substantially 10 millimeters (mm). It is contemplated, however, that the height 124 may be varied according to the bone joint to be treated, and thus the implant 100 may be implemented with a wide variety of heights 124, without limitation.

The cylindrical sidewall 112 of the implant 100 includes a taper that causes a diameter of the sidewall 112 to decrease from a diameter of the top portion 104 to the bottom diameter 132 of the bottom portion 108. As shown in FIG. 4, the taper of the sidewall 112 may be expressed in terms of a taper half-angle 136 taken with respect to the longitudinal axis 128. The taper of the sidewall 112 is configured to prevent the implant 100 from subsiding into the osteochondral hole drilled in bone. As will be appreciated, therefore, the taper half-angle 136 may be any angle that is found to prevent subsidence of the implant 100, without limitation. Accordingly, it is contemplated that in some embodiments, the taper half-angle 136 is substantially zero degrees. In such embodiments, the diameter of the top portion 104 is substantially the same as the bottom diameter 132 of the bottom portion 108, and thus the cylindrical sidewall 112 comprises a straight cylindrical shape, without limitation.

In some embodiments, the overall size of the implant 100 is identified based on the bottom diameter 132 without a specific reference to the included taper half-angle 136 of the implant 100. In such embodiments, a practitioner may select the implant 100 based on a size of the osteochondral hole to be drilled into the patient's bone. As with other dimensions of the implant 100 discussed hereinabove, however, the bottom diameter 132 may be varied according to the bone joint to be treated. In one embodiment, the bottom diameter 132 ranges between substantially 5 mm and substantially 10 mm. As will be appreciated, therefore, the implant 100 may be implemented with a wide variety of bottom diameters 132, without limitation.

In some embodiments, the overall size of the implant 100 may be identified based on the diameter of the top portion 104, and thus the size of the implant 100 may be selected based on the area of the joint defect to be treated. It is contemplated that in such embodiments, the specific sizes of the bottom diameter 132 and the taper half-angle 136 may be incorporated into the implant 100 in accordance with the diameter of the top portion 104, and thus the sizes of the bottom diameter 132 and the taper half-angle 136 need not be specifically called out. For example, in some embodiments, any one or more of the height 124, the taper half-angle 136, and the bottom diameter 132 of the implant 100 may be configured to correlate with the diameter of the top portion 104, without limitation.

FIG. 5 illustrates an exemplary use environment wherein the tapered monophasic implant 100 is implanted into an osteochondral hole 140 drilled in a 1^{st} metatarsal bone 144. As will be recognized, the top portion 104 of the implant 100 is disposed slightly above the surrounding cartilage tissue of the 1^{st} metatarsal bone 144 and in contact with an adjacent 1^{st} proximal phalangeal bone 148. In general, the top portion 104 includes a shape configured to approximate the osteochondral surface to be replaced. In some embodiments, such as the illustrated embodiment of FIG. 5, the shape of the top portion 104 includes a convex curvature that approximates the curvature of the osteochondral surface to be replaced. In embodiments of the top portion 104 including a convex curvature, the implant 100 includes a positive curvature height 152 as shown in FIG. 4. In some embodiments, the top portion 104 includes a concave curvature that corresponds to a negative curvature height 152 of the implant 100. It is contemplated that an embodiment of the implant 100 including a negative curvature height 152 is advantageously configured for treating cartilage defects in the 1^{st} proximal phalangeal bone 148.

As further shown in FIG. 5, the implant 100 includes a height 124 that places the bottom portion 108 in contact with a bottom of the osteochondral hole 140 and elevates the top portion 104 slightly above the surrounding cartilage tissue of the 1^{st} metatarsal bone 144. The taper half-angle 136 advantageously prevents subsidence of the implant 100 into the osteochondral hole 140, even in the event that the bone below the bottom portion 108 subsides. As best illustrated in FIG. 4, the implant 100 includes a rounded periphery 156 that joins the top portion 104 and the cylindrical sidewall 112. The rounded periphery 156 comprises a transition surface between the top portion 104 and the sidewall 112 that provides a smooth contact surface to surrounding tissues. Further, the implant 100 includes a rounded periphery 160 that joins the cylindrical sidewall 112 and the bottom portion 108. As will be appreciated, the rounded periphery 160 provides a smooth transition surface between the sidewall 112 and the bottom portion 108 that prevents damage to the interior sidewalls of the osteochondral hole 140 during insertion of the implant 100 therein.

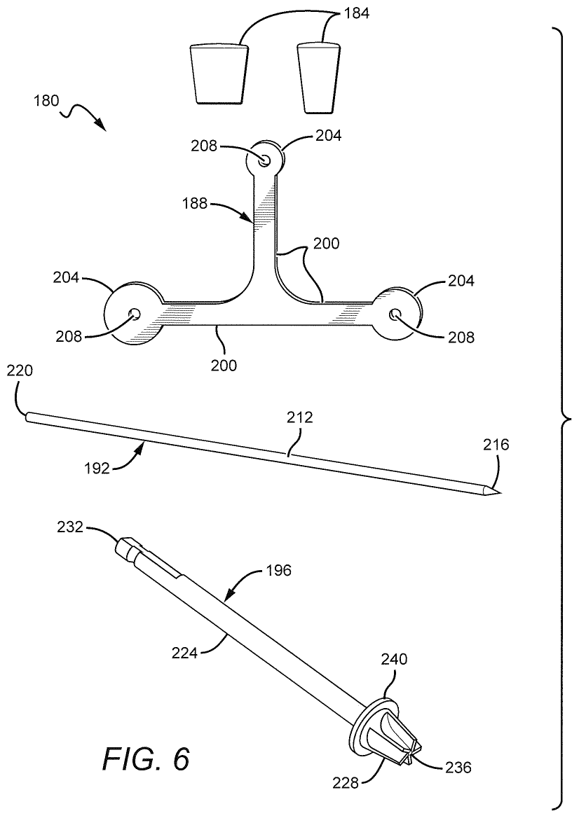

FIG. 6 illustrates an exemplary embodiment of a sterile instrument kit 180 for treating damaged cartilage joints according to the present disclosure. In the embodiment illustrated in FIG. 6, the instrument kit 180 comprises one or more tapered osteochondral implants 184, a size gauge 188, a guidewire 192, and a cannulated reamer 196. In some embodiments, the instrument kit 180 may further comprise a graft inserter and/or a tamp (not shown). As will be appreciated, the instrument kit 180 comprises instruments necessary to for treating osteochondral defects by way of surgery. The sizes of the instruments comprising the kit 180 will depend upon the size of the particular implant 184 to be implanted into the patient. It is envisioned, therefore, that a surgeon may select the implant 184 and a correspondingly sized embodiment of the instrument kit 180 based on the location and size of the bone joint to be treated.

With continuing reference to FIG. 6, the size gauge 188 comprises multiple tabs 200, each of which representing a particular size of the implant 184. Each of the multiple tabs 200 includes a circular portion 204 having a central hole 208. The circular portions 204 approximate the areas of different implants 184, and the central hole 208 has a diameter suitable to receive the guidewire 192. As will be appreciated by those skilled in the art, the circular portions 204 facilitate identifying an advantageously sized implant 184 for treating the damaged bone joint. The central hole 208 facilitates inserting the guidewire 192 through the size gauge 188.

The guidewire 192 comprises an elongate shaft 212 having a distal pointed tip 216 and a proximal blunt end 220. The guidewire 192 is configured to be inserted into confined spaces within bone joints and serves to direct a subsequent insertion of the cannulated reamer 196 to the implant location within the bone joint. In some embodiments, the guidewire 192 is comprised of a surgical stainless steel, such as austenitic 316 stainless steel, martensitic 440 stainless steel, martensitic 420 stainless steel, and the like. It will be appreciated that the distal pointed tip 216 facilitates advancing the guidewire 192 through obstructive tissues and structures, and the proximal blunt end 220 facilitates manipulating the guidewire 192 by hand, or by way of an appropriate tool.

The cannulated reamer 196 comprises a rigid elongate shaft 224 having a distal cutting end 228 and a proximal shank 232. The distal cutting end 228 comprises a cutting edge suitable for rotatably clearing a tapered osteochondral bore, thereby removing damaged articular cartilage and an underlying bone portion from the bone joint being treated. In some embodiments, the distal cutting edge 228 comprises a spiral cutting edge, although other suitable cutting edge configurations will be apparent. The proximal shank 232 is configured to be grasped by a chuck of a surgical drill, or other equivalent rotary tool. Further, in some embodiments the cannulated reamer 196 comprises a central, lengthwise hole 236 whereby the reamer may be mounted onto the guidewire 192 so as to direct the distal cutting end 228 to the damage location within the bone joint. A peripheral disc 240 is configured to operate as a depth gauge. As will be appreciated, the disc 240 coming into contact with tissue surround a bore being drilled operates as an indication that the bore has an optimal depth to receive the tapered implant 184.

It is contemplated that, in some embodiments, the distal cutting edge 228 includes a tapered diameter that corresponds to the tapered diameter of the implant 184, as described herein. In general, the shape and size of the distal cutting edge 228 included in the instrument kit 180 corresponds the shape and size of the particular implant 184 included in the kit, as well as being indicated by at least one of the circular portions 204 of the size gauge 188. Thus, it is contemplate that the surgeon may use the size gauge 188 to select an advantageously sized implant 184 to replace damaged cartilage in the bone joint, and then extend the guidewire 192 through the central hole 208 to locate a center of the bore to be drilled. With the size of the implant 184 known, the surgeon may remove the size gauge 188 from the guidewire 192 and then extend an appropriately sized cannulated reamer 196 along the guidewire 192 to the site of the damaged cartilage to be removed. Other surgery techniques will be apparent to those skilled in the art.

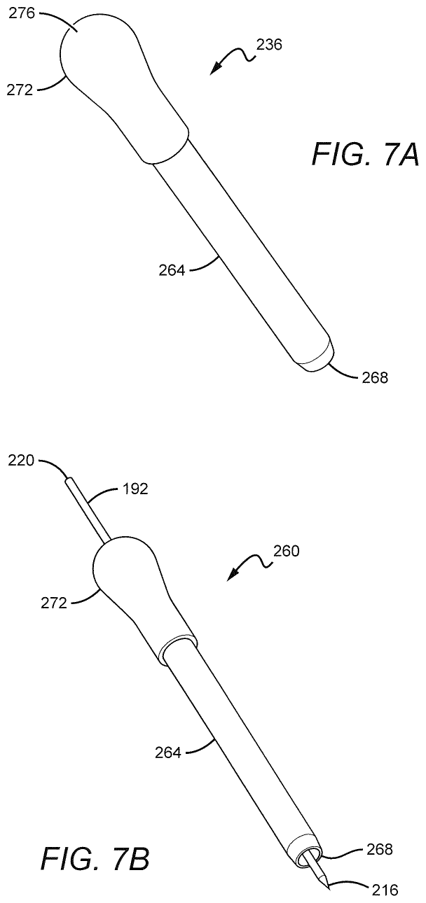

FIG. 7A illustrates an exemplary embodiment of a punch 260 that may, in some embodiments, be included in the instrument kit 180 shown in FIG. 6. The punch 260 comprises a generally elongate member 264 having a distal punch blade 268 and a rounded proximal handle 272. The distal punch blade 268 comprises a cutting edge suitable for stamping a shaped cut into the cartilage prior to drilling with the cannulated reamer 196 as described above. The shaped cut facilitates removing damaged articular cartilage from the bone joint being treated. In some embodiments, the distal punch blade 268 is circular, and thus enables stamping a circular cut in the cartilage. Shapes other than circular are contemplated, however, such as, by way of non-limiting example, any of various generally circular, oval, round, or other closed perimeter shapes, and the like, without limitation. The rounded proximal handle 272 is configured to be grasped by hand for pushing the distal punch blade 268 into the cartilage for cutting purposes. Further, the punch 260 comprises a central, lengthwise hole 276. As best shown in FIG. 7B, the hole 276 enables the punch 260 to be mounted onto the guidewire 192 so as to direct the distal punch blade 268 to the damage location within the bone joint.

It is envisioned that the instrument kit 180 is to be suitably sterilized for surgeries, and packaged into sterilized containers. In some embodiments, the size gauge 188 is packaged in a first sterile container, while the guidewire 192, the cannulated reamer 196, the punch 260, and a graft inserter, if included, are packaged in a second sterile container, and the tapered implant 184 is packaged in a third sterile container. The first, second, and third sterile containers are then bundled together into a single, exterior container, thereby forming a convenient surgery-specific cartilage repair package. It is envisioned that other packaging techniques will be apparent to those skilled in the art without deviating from the spirit and scope of the present disclosure.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. An implant for treating osteochondral defects, comprising:
a cylindrical member comprised of a monophasic synthetic material;
a top portion comprising a first diameter;
a bottom portion comprising a second diameter; and
a tapered sidewall portion disposed between the top portion and the bottom portion.

2. The implant of claim 1, wherein the tapered sidewall portion includes a diameter that decreases from the first diameter to the second diameter.

3. The implant of claim 2, wherein the tapered sidewall portion comprises a degree of tapering that is configured to prevent the implant from subsiding into the hole drilled in bone.

4. The implant of claim 1, wherein the implant includes a surface area ranging between substantially 6 square inches and substantially 17 square inches.

5. The implant of claim 1, wherein the monophasic synthetic material includes any one of silicone, bioglass, peek, polyethylene, titanium, and cobalt chrome.

6. The implant of claim 1, wherein the first diameter and the second diameter are selected according to a location in a patient that is to be treated.

7. The implant of claim 6, wherein a height of the cylindrical member is substantially 10 millimeters (mm), and the first diameter ranges between substantially 5 mm and substantially 10 mm.

8. The implant of claim 1, wherein the top portion includes a shape configured to approximate an osteochondral surface to be replaced.

9. The implant of claim 8, wherein the shape includes a curvature of the top portion that approximates the curvature of the osteochondral surface to be replaced.

10. The implant of claim 9, wherein the curvature is either convex or concave so as to match the anatomy of the osteochondral surface.

11. The implant of claim 1, further comprising a rounded periphery that joins the top portion and the tapered sidewall portion, the rounded periphery providing a smooth contact surface to surrounding tissues.

12. The implant of claim 1, further comprising a rounded periphery that joins the tapered sidewall portion and the bottom portion, the rounded periphery providing a smooth transition surface between the tapered sidewall portion and the bottom portion.

13. A sterile instrument kit for repairing osteochondral defects, the instrument kit comprising:
one or more tapered implants configured to treat osteochondral defects in various bone joint locations in a patient's body, the one or more tapered implants each comprising monophasic synthetic material; and
a multiplicity of instruments including any one or more of an implant inserter, a guidewire, a cannulated reamer, a punch, and a size gauge, the multiplicity of instruments being configured for implanting the one or more tapered implants into the patient's body such that the implant is flush or slightly proud of a surrounding native cartilage surface.

14. The instrument kit of claim 13, wherein the one or more tapered implants and the multiplicity of instruments are packaged together in an exterior container suitable for delivery to a practitioner.

15. The instrument kit of claim 14, wherein the one or more tapered implants are stored in a first sterile container.

16. The instrument kit of claim 15, wherein any one or more of the guidewire, the cannulated reamer, the punch, and the implant inserter are stored in a second sterile container.

17. The instrument kit of claim 16, wherein the size gauge is stored in a third sterile container.

18. A method for a sterile instrument kit for repairing osteochondral defects, comprising:
configuring one or more tapered implants to treat osteochondral defects in various bone joint locations in a patient's body; and
combining the one or more tapered implants with a multiplicity of instruments configured for implantation of the one or more tapered implants into the patient's body, the multiplicity of instruments including at least a guidewire, a cannulated reamer, a punch, and a size gauge.

19. The method of claim 18, wherein configuring comprises forming the one or more tapered implants of a monophasic synthetic material, including any or one of silicone, bioglass, peek, polyethylene, titanium, and cobalt chrome.

20. The method of claim 18, wherein configuring comprises foil ling the one or more tapered implants such that the diameters of a top portion of the one or more tapered implants range from substantially 5 mm to substantially 10 mm.

21. The method of claim 20, wherein combining further comprises:
storing the one or more tapered implants in a first sterile container;
storing any one or more of the guidewire, the cannulated reamer, the punch, and an implant inserter in a second sterile container; and
storing the size gauge in a third sterile container.
